(19) United States
(12) Patent Application Publication
(10) Pub. No.: US 2026/0325196 A1
(43) Pub. Date: Oct. 22, 2026

(54) INTEGRATED INFUSION AND ABLATION SYSTEM AND METHOD

(71) Applicant: Baylis Medical Technologies Inc., Mississauga (CA)

(72) Inventor: Neil Godara, Milton (CA)

(21) Appl. No.: 19/575,285

(22) Filed: Mar. 23, 2026

Related U.S. Application Data
(60) Provisional application No. 63/775,534, filed on Mar. 21, 2025.

(51) Int. Cl. A61B 18/12; A61B 18/00

(52) U.S. Cl. CPC A61B 18/1206; A61B 2018/00011; A61B 2018/00666; A61B 2018/00791

(57) ABSTRACT

A system and methods for ablating soft tissue is disclosed. The system includes an electrosurgical ablation device connected to a generator, a fluid injection device, a fluid delivery device, and at least one sensor. During an ablation procedure, fluid delivery to a target tissue is automatically controlled based on measurements from a sensor. An exemplary disclosed method of using the system includes the steps of: inserting a electrosurgical ablation device and a fluid delivery device to a target location within a patient's body, monitoring at least one tissue parameter, detecting whether the value is within an allowable range, if the parameter is within an allowable range, delivering radiofrequency energy to tissue, and if the parameter exits the allowable range, delivering fluid to the tissue.

### TECHNICAL FIELD

The disclosure relates to medical devices, and more particularly to devices, systems, and methods for ablating tissue using radiofrequency energy in combination with fluid injection.

### BACKGROUND OF THE ART

The ablation of benign thyroid nodules has been touted as a possible replacement for surgical resection of the thyroid gland, for symptomatic benign thyroid nodules. While there are several benefits to percutaneous ablation when compared to surgical resection, such as better preservation of thyroid function and improved aesthetic outcomes, there are inherent limitations and risks when using radiofrequency ablation. In particular, thyroid nodules are often located close to sensitive neural structures, such as the recurrent laryngeal nerve. Other structures of interest also surround thyroid nodules (e.g. esophagus, carotid artery, vocal cords) and must also be avoided. When ablating tissue, it may not be possible to predict the extent of the ablation volume. Current techniques to protect sensitive structures during thyroid ablation include using ablation probes that produce smaller ablation volumes. In some instances, this may not be practical, or may not provide a sufficient safety margin.

Because of this, physicians use injections of fluids in the surrounding tissue for a multitude of reasons. In some instances, physicians choose to displace the benign nodule from sensitive structures through a technique called “hydro dissection”. In this technique, a mix of dextrose (5%) and water (95%), sometimes referred to as D5W, is injected in the inter-capsule space between the nodule and the sensitive structures to be protected during ablation. D5W is used as it does not conduct electricity in the way that normal saline would. As such, it not only physically separates the ablation zone from the sensitive structures, but it also provides an electrical and thermal isolation barrier as well.

It is normal for the D5W to dissipate during the procedure, necessitating subsequent D5W injections. In other instances, physicians may continuously deliver D5W to the desired area by connecting a needle (for example, a spinal needle) to a peristaltic pump.

In some instances, D5W hydro-dissection may not be possible or not preferred. In these cases, physicians may monitor the patient to determine if sensitive structures have been compromised during the ablation of the benign thyroid nodule. For example, physicians require a patient to be awake during the ablation procedure so that they can vocalize upon request. Physicians will monitor the tone and quality of the patient's voice, and if a change in tone is identified, injury to the recurrent laryngeal nerve would be suspected. If this happens, immediate corrective action is required, typically involving removal of the ablation system and the injection of cold saline to the suspected injured area to prevent further nerve damage or to protect the nerve. In all instances, it is incumbent on the physician to continuously monitor the sensitive neural structures in the vicinity of the ablation zone and perform measures to either prevent or reverse damage that may occur.

The ability to ablate a portion of tissue with RF energy, and automatically inject fluids to a second location to protect sensitive structures before damage occurs could offer advantages.

### BRIEF DESCRIPTION OF THE DRAWINGS

In order that the invention may be readily understood, embodiments of the invention are illustrated by way of examples in the accompanying drawings, in which:

FIG. 1 is an illustration of a system for delivering radiofrequency energy and fluid to a region of tissue in accordance with an embodiment of the present invention;

FIG. 2 is an illustration of an RF energy delivery device in accordance with an embodiment of the present invention;

FIGS. 3A and 3B are illustrations of the distal region of the device of FIG. 2, and corresponding ablation zones, in accordance with an embodiment of the present invention;

FIG. 4 is a cross-section illustration of the distal region of the RF device in accordance with an embodiment of the present invention;

FIG. 5 is an illustration of the distal region of the RF device in accordance with an embodiment of the present invention;

FIG. 6A is an illustration of a fluid injection device in accordance with an embodiment of the present invention;

FIG. 6B is a cross-section illustration of a fluid injection device in accordance with an embodiment of the present invention;

FIG. 7 is a flow diagram showing a method of ablating tissue and injecting fluid in accordance with an embodiment of the present invention;

FIGS. 8A to 8C illustrate a method of ablating tissue and injecting fluid in accordance with an embodiment of the present invention;

FIG. 9 is a flow diagram showing a method of ablating tissue and injecting fluid in accordance with an alternative embodiment of the present invention; and

FIGS. 10A to 10C illustrate a method of ablating tissue and injecting fluid in accordance with an alternative embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

To overcome the problems described herein, the inventors have developed a novel integrated ablation and fluid delivery system and corresponding method. The system includes a fluid injection device that optimizes and automates many parts of the thyroid ablation procedure.

At a high level, a fluid injection device (e.g., needle) with an integrated thermocouple is connected to a peristaltic pump. In some instances, the pump and the needle form part of the ablation system. In cases where hydro-dissection is required, the pump can be controlled directly through the same interface as the ablation system. This has the benefit of limiting injection during the period when RF ablation is active and reduces the need for an independent pump for this function. In order to avoid injury to nerves, the needle with integrated thermocouple is used to continuously monitor the temperature in an area of interest.

For example, this could be in proximity to the sensitive structures that are to be protected during the ablation procedure. When a temperature rise is measured, for example 42C, which is just below the threshold for neural injury, the pump that is connected to the needle can be triggered to automatically inject fluid around the sensitive structures. In this way, the mechanism is preventative (prior to recurrent laryngeal nerve injury) rather than corrective (injection of fluid on the nerve after a change in tone of voice).

In one embodiment, the system uses a bolus mode, which may be activated by the user through buttons on the handheld ablation device. In some embodiments, the bolus mode, continuous mode, or temperature-initiated bolus mode are activated or affected by images gathered by an ultrasound system. In such an embodiment, special features in the imaging features such as the loss of spaces between the thyroid nodule margin and the sensitive structures automatically trigger injection of fluid through this final needle.

In some embodiments, the system is preferentially coupled or integrated with a neuromonitoring system. In some examples, the needle may have the ability to deliver stimulus pulses to the surrounding tissue. In other examples, a stimulation pulse is delivered by an independent electrode or external device. The neuromonitoring system is configured to detect the pulses, and potential changes in nerve response, and is further configured to communicate with the fluid injection system to deliver a bolus of fluid or continuous fluid if nerve damage is suspected. In some examples, the neuromonitoring and stimulation features are integrated with the radiofrequency ablation system.

In some embodiments, the system is configured to halt RF energy delivery to the RF ablation probe upon detection of the hazardous situation. Optionally, the rate of coolant flow to the RF ablation probe can be maintained or increased, for a probe design to receiving coolant flow, when radiofrequency energy is halted to further support a reduction in tissue temperature in the ablation zone. This would cooperatively affect the safety outcome of the primary invention.

The system can work with any energy delivery source that is intended ablate tissue with sensitive structures that need to be protected.

In one broad aspect, embodiments of the present invention comprise a method of delivering energy to a region of tissue within a patient's body in conjunction with controlled fluid delivery, the method comprising the steps of: monitoring a tissue parameter within the region of tissue; detecting whether a value of the tissue parameter is within an allowable range; while the tissue parameter remains within the allowable range, delivering radiofrequency energy to a first portion of tissue within a region of tissue; if the tissue parameter exits the allowable range, automatically delivering fluid to the region of tissue.

As a feature of this aspect, fluid is delivered between the first portion of tissue within the region of tissue and a second portion of tissue within the region of tissue

As a feature of this aspect, the tissue parameter is a temperature.

As a feature of this aspect, the allowable range is less than 42 degrees Celsius.

As a feature of this aspect, the tissue parameter is a distance between the first portion of tissue and the second portion of tissue.

As a feature of this aspect, the step of delivering radiofrequency energy is performed using a first device and step of injecting fluid is performed using a second device.

As a feature of this aspect, the step of monitoring the tissue parameter is performed using the second device.

As a feature of this aspect, the first device is a radiofrequency ablation device, and the second device comprises a needle.

As a feature of this aspect, the second device comprises a temperature sensor.

As a feature of this aspect, the second device comprises an injection port.

As a feature of this aspect, the method further comprises a step of cooling a portion of the radiofrequency ablation device.

As a feature of this aspect, the radiofrequency energy is transmitted by an ablation device, while the ablation device is operating in a monopolar mode.

As a feature of this aspect, the radiofrequency energy comprises a bipolar energy delivery profile.

As a feature of this aspect, the step of monitoring the tissue parameter is performed within a third portion within the region of tissue, the third portion located between the first portion and the second portion.

As a feature of this aspect, the tissue parameter is a temperature.

As a feature of this aspect, the method further includes a step of monitoring a first temperature in the first portion of tissue, and the tissue parameter is a second temperature located between the first portion of tissue and the second portion of tissue.

As a feature of this aspect, the method further comprises a step of comparing the first temperature and the second temperature.

As a feature of this aspect, the step of monitoring the first temperature is performed with a first device, and monitoring the second temperature is performed with a second device.

As a feature of this aspect, the fluid is delivered for a predetermined amount of time.

As a feature of this aspect, delivering fluid includes delivering a predetermined volume of fluid.

As a feature of this aspect, the region of tissue is a thyroid, and the first portion of tissue is a thyroid nodule.

As a feature of this aspect, the tissue parameter is a first tissue parameter, the value is a first value, and the allowable range is a first allowable range, the method further comprises the steps of: monitoring a second tissue parameter within the region of tissue; detecting whether a second value of the second tissue parameter is within a second allowable range; while the second tissue parameter remains within the second allowable range, delivering radiofrequency energy to the first portion of tissue within a region of tissue; if the second tissue parameter exits the second allowable range, automatically delivering fluid to the region of tissue.

In another broad aspects, embodiments of the present invention include a system for controlling delivery of radiofrequency energy and controlling delivery of fluid to a region of tissue comprising: an electrosurgical device for delivering radiofrequency energy; a fluid injection device; a fluid delivery device; and a generator in communication with the electrosurgical device, the fluid injection device, and the fluid delivery device; the generator monitors at least one tissue parameter, and wherein the generator controls delivery of radiofrequency energy to the electrosurgical device and controls delivery of fluid to the fluid injection device in response to a change in a value of the at least one tissue parameter.

As a feature of this aspect, the electrosurgical device comprises a bipolar radiofrequency ablation device.

As a feature of this aspect, the electrosurgical device comprises a monopolar radiofrequency ablation device.

As a feature of this aspect, the electrosurgical device comprises an elongate member defining a lumen for circulating a cooling fluid, and wherein the generator controls circulation of the cooling fluid.

As a feature of this aspect, the electrosurgical device comprises at least one temperature sensor.

As a feature of this aspect, the fluid injection device comprises a temperature sensor, and the at least one tissue parameter is a temperature value.

As a feature of this aspect, the system further comprises an ultrasound imaging device, and the at least one tissue parameter is a distance between a first portion of tissue within the region of tissue and a second portion of tissue within the region of tissue, wherein the distance is measured using ultrasound imaging.

As a feature of this aspect, the system further comprises an ultrasound imaging device, and the generator monitors a first tissue parameter and a second tissue parameter, wherein the first tissue parameter is a temperature value and the second tissue parameter is a distance between a first portion of tissue within the region of tissue and a second portion of tissue within the region of tissue, wherein the distance is measured using ultrasound imaging.

As a feature of this aspect, the generator comprises software, the software including instructions for monitoring a tissue parameter and directing the fluid delivery device to deliver fluid to the fluid injection device.

As a feature of this aspect, the software includes instructions to modify the delivery of radiofrequency energy in response to the change in the value of the at least one tissue parameter.

As a feature of this aspect, the system further comprises a fluid deactivation mechanism.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

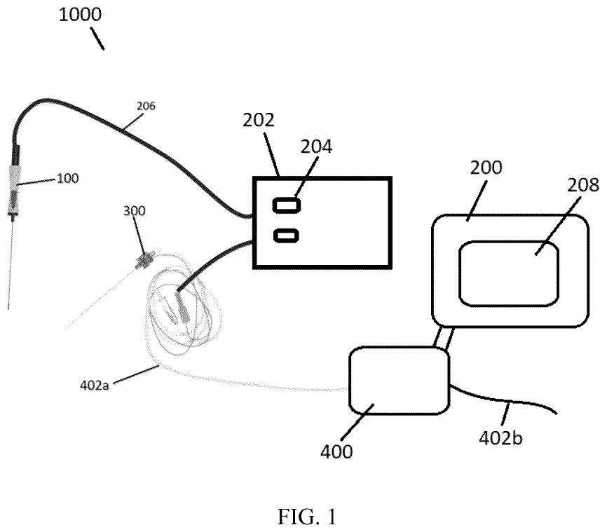

With reference to FIG. 1, an example tissue ablation system 1000 is shown. Tissue ablation system 1000 generally includes a radiofrequency ablation device 100 for delivering ablative energy to target tissue. Tissue ablation system 1000 further includes a radiofrequency (RF) generator 200 for delivering the ablative energy to an active distal region of the radiofrequency ablation device 100. In some embodiments, radiofrequency ablation device 100 is a monopolar probe and the system 1000 includes a return electrode in the form of a grounding pad (not shown), for returning a return current from the active distal region to the radiofrequency generator 200. In other embodiments, radiofrequency ablation device 100 is a bipolar probe, and energy is delivered through an active electrode and a return electrode.

RF generator 200 can be, for example, a generator that outputs 50 W to 100 W, and has a control system capable of modulating the RF energy output based on at least one measurable parameter of the radiofrequency ablation device 100 (e.g. temperature). In some embodiments, generator 200 is coupled with a hub 202 that includes a plurality of ports 204. Hub 202 is configured to form an electrical connection with a plurality of devices, for example radiofrequency ablation device 100 and fluid injection device 300, via one or more electrical cables 206 connected to the plurality of ports 204.

Generator 200 further comprises user controls 208 which includes a user interface, for example a touchscreen, and/or one or more buttons, switches, knobs, etc., that allow control of various settings or parameters of the generator and connected devices. For example, user controls 208 can activate/deactivate delivery RF energy delivery, start or stop cooling to the probe or fluid delivery to the fluid injection device. Generator 200 includes software that allows for continuous monitoring of various parameters described herein.

System 1000 further includes a fluid injection device 300 in fluid communication with a fluid delivery device 400 and in communication with generator 200. Fluid injection device 300 may be any elongate device, for example a needle, capable of injecting a fluid into tissue. In some embodiments, fluid injection device 300 comprises one or more sensors, electrically connected to generator 200, to relay sensor information to generator 200. Generator 200 then uses information obtained from one or more sensors to control the fluid delivery device 400.

Fluid delivery device 400 can be, for example, a pump 400 that pumps fluid through tubing and into fluid injection device 300, which exits through a distal opening. In some embodiments, radiofrequency ablation device 100 includes cooling features, described herein, and fluid delivery device 400 pumps (circulates) cooling fluid to radiofrequency ablation device 100. Fluid delivery device 400 comprises a plurality of fluid delivery tubes 402 for connecting to the device and connecting to an external fluid source (not shown). For example, fluid tube 402a connected to fluid injection device 300, and fluid tube 402b connected to an external fluid source.

The fluid delivery device 400 is usable with the RF generator 200 and configured to deliver fluid to a plurality of devices in response to commands from generator 200.

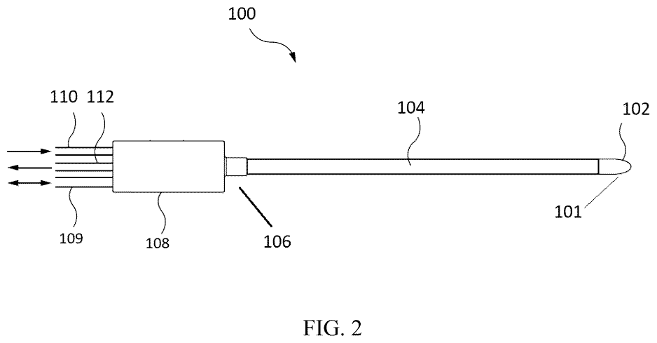

With reference to FIG. 2, in one embodiment, radiofrequency ablation device 100 (or “probe”) comprises an elongate member or shaft 104 having an active distal region 102 and a proximal end 106. Handle 108 is coupled to shaft 104 proximal end 106.

In one embodiment, radiofrequency ablation device 100 is insulated along its shaft 104 to prevent ablation outside of the target site. Radiofrequency ablation device 100 is configured to be inserted through the skin to the target site where soft tissue is to be lesioned. In some embodiments, radiofrequency ablation device 100 comprises a conductive shaft and further comprises an insulating layer. In this embodiment, energy is delivered via at least one active portion (e.g., an exposed portion of the conductive shaft). In another embodiment, radiofrequency ablation device 100 comprises a non-conductive shaft 104 and energy is delivered via a conductive wire (not shown), coupled to the active portion located on the radiofrequency ablation device 100. In such an embodiment, the conductive wire would couple the active portion (e.g., an electrode) to the RF generator 200.

In one embodiment, a handle 108 at its proximal end which accommodates an electrical connection to the RF generator 200. Generator 200 is connected to the probe handle 108 by cable/wire 109. Generator 200 supplies radiofrequency ablation device 100 with high frequency energy such as radiofrequency which is transmitted to the active distal region 102 at the target site. The high frequency energy heats the tissue at the target site and forms a lesion by thermal ablation.

In one embodiment, radiofrequency ablation device 100 is a “cooled probe”, i.e., it comprises cooling features, and handle 108 provides a connection to the fluid delivery system 300 through cooling tubes 110 and 112. Elongate member 104 defines a lumen for circulating fluid, described herein.

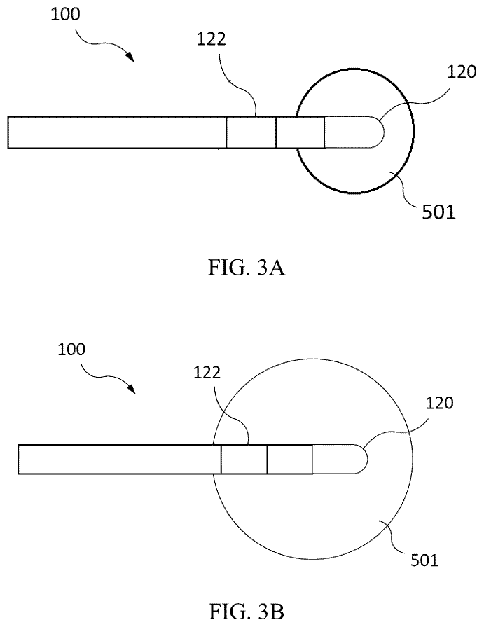

FIG. 3A shows a close-up view of active distal region 102. In one embodiment, active distal region 102 comprises a first electrode 120 and a second electrode 122. Second electrode 122 may be positioned along shaft 104 proximal to active distal region 102 forming a coaxial, bipolar, probe.

In one embodiment, radiofrequency ablation device 100 delivers energy through one of the two electrodes 120 or 122, and radiofrequency ablation device 100 is used with a return electrode such as a grounding pad (not shown). The return electrode is placed in conductive contact with skin and is connected to generator 200. The return electrode completes the electrical circuit for the high frequency energy through the patient's body. FIG. 3A shows ablation zone 501 created using first electrode 120 in a monopolar mode. FIG. 3B shows the probe being used in a bipolar mode, where one of electrodes (e.g., 120) is used as an active electrode, and the other (e.g., 122) is used as a return electrode, and a second, larger ablation zone 501 is created.

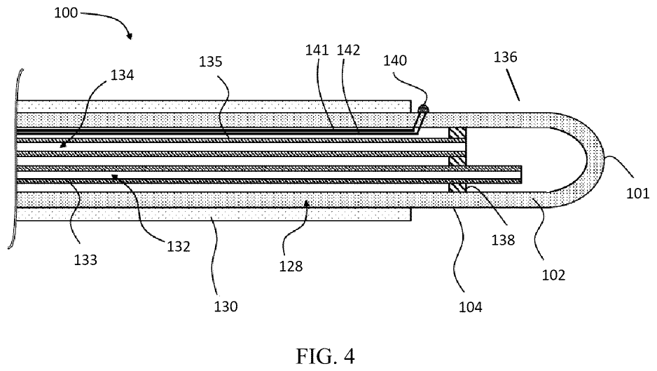

In one embodiment, radiofrequency ablation device 100 is a cooled probe, described herein, and comprises cooling features. FIG. 4 shows a cross section of shaft 104 distal region. In one embodiment, radiofrequency ablation device 100 includes a fluid delivery lumen 132 for delivering cooling fluid through the shaft 104 to a cooled section 136, and a fluid return lumen 134 for delivering the cooling fluid out of the cooled section 136 and proximally out of the shaft 104. In the example shown, the fluid delivery lumen 132 is provided by a first hypotube 133 and the fluid return lumen is provided by a second hypotube 135. The first and second hypotubes 133, 135 can be connected to cooling tubes 110 and 112, which in turn are connected to the fluid delivery device 400. The delivery of the cooling fluid through to the cooled section 136 cools at least a portion of the active distal region 102.

In some embodiments, the shaft 104 is configured so that on delivery of the cooling fluid (i.e. upon the exit of the cooling fluid from the fluid delivery lumen 132 and prior to the entry of the cooling fluid into the fluid return lumen 134), the cooling fluid is restricted to the cooled section 136 which is defined by barrier 138. Radiofrequency ablation device 100 further comprises insulation 130 covering a portion of shaft 104 and defining an insulated region 128. In one embodiment, barrier 138 is positioned distally of the insulated region 128. In other embodiments, barrier 138 is located within the insulated region 128 or at the distal end of the insulated region 128.

In some embodiments, radiofrequency ablation device 100 includes at least one temperature sensor 140 to measure the temperature of the tissue in or near an ablation zone. The temperature sensor 140 can be, for example, a thermocouple junction, and the radiofrequency ablation device 100 can further include a pair of electrical conductors 141, 142 for electrically connecting the thermocouple junction to a temperature control system (which can be built into the generator 200) via electrical cable 206. In alternative examples, temperature sensor 140 can be another type of temperature sensor, such as thermistor, a thermometer, or an optical fluorescence sensor. In the example shown, the electrical conductors 141, 142 extend through a lumen of the shaft 104.

Temperature sensor 140 is positioned to measure a temperature in a heat affected zone of the target tissue, and more specifically, in the thermal ablation zone. That is, the temperature sensor 140 is not internal to the shaft 104, but rather, is external to shaft 104 so that in use it is in contact with the target tissue and is sufficiently close to the active distal region 102 to measure the temperature in the thermal ablation zone.

Further, temperature sensor 140 can be positioned at distal end 101 or proximal of the distal end 101. In one embodiment, the temperature sensor 140 is affixed to one of the electrodes 120 or 122, for example by laser welding. In other embodiments, radiofrequency ablation device 100 comprises a plurality of temperature sensors 140.

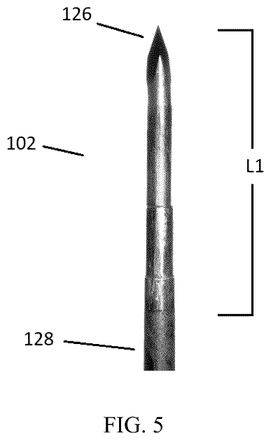

Referring to FIG. 5, in one embodiment, radiofrequency ablation device 100 comprises a sharp tip 126, also referred to as a “trocar tip”, to help facilitate delivery of the radiofrequency ablation device 100, through a patient's skin, to the treatment location. In one embodiment, trocar tip 126 comprises radiopaque features so it is visible using one or more imaging modalities. In other embodiments, shaft 104 comprises radiopaque features (not shown) at other locations, for example marker bands. Active distal region 102 spans a length of L1. In some examples, L1 is between about 5 mm and about 15 mm. In some embodiments trocar tip 126, active distal region 102, and/or other portions of the shaft 104 comprise one or more echogenic features. Echogenic features may be either passive (features on the electrode surface that reflect ultrasound energy back to the transducer), or active (an energy source is coupled to the electrode that sends acoustic energy that can be detected by the transducer).

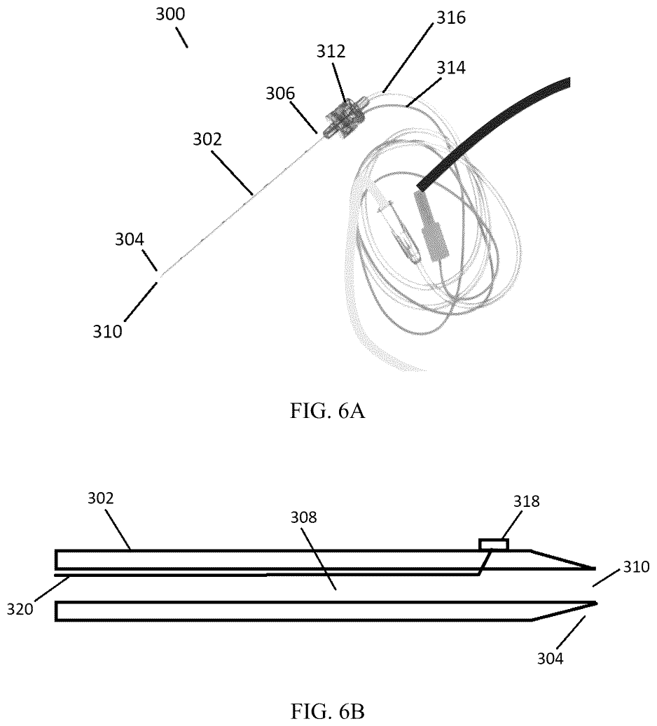

With reference to FIGS. 6A and 6B, in one embodiment, fluid injection device 300 is a needle comprising an elongate member 302 having a distal end 304 and a proximal end 306. Elongate member 302 defines lumen 308 extending from the proximal end 306 to the distal end 304 and terminating at a distal opening 310.

Elongate member 302 is coupled to a needle hub 312 at proximal end 306. Needle hub 312 is coupled to at least one electrical cable (or “wire”) 314, and a fluid tube 316. Electrical cable 314 connects to generator 200 or generator hub 202, and fluid tube 316 connects to fluid delivery device 400. Fluid delivery device 400 pumps fluid through fluid tube 316, into lumen 308, such that it exits distal opening 310 and is delivered into a region of tissue.

In one embodiment, fluid injection device 300 comprises a temperature sensor 318 located at or near distal end 304, for example in a distal region. In one embodiment, temperature sensor 318 is a wireless sensor. In another embodiment, temperature sensor 318 is connected via one or more wires 320 for electrically connecting the temperature sensor 318 to the needle hub 312 and/or electrical cable 314, such that a temperature parameter measured by the temperature sensor can be sent to generator 200. In one embodiment, temperature sensor 318 comprises a thermocouple.

Fluid delivery device 400 may be any device, for example a pump, capable of pumping fluid to one or more devices. In one embodiment, fluid delivery device 400 has a plurality of ports, for connecting fluid tubes 110, 112, and 316, and is electrically coupled to generator 200. Generator 200 instructs fluid delivery device 400 to send fluid to the fluid injection device 300. In some embodiments, radiofrequency ablation device 100 comprises cooling features, described herein and pump 400 delivers cooling fluid to radiofrequency ablation device 100. (NOTE: fluid tubes 110, 112 connecting radiofrequency ablation device 100 to fluid delivery device 400 are not shown in FIG. 1.)

In some embodiments, generator 200, in combination with a temperature control system (not shown), can automatically activate or deactivate the cooling, and/or adjust the cooling rate (flow rate), in response to measured temperature values in the ablation zone. For example, when certain threshold temperatures are met, cooling may turn on or turn off, or the flow rate may be modified automatically by the generator. In other embodiments, cooling may be manually activated, deactivated, or flow rate may be manual modified by a user using one or more switches, described herein.

In some embodiments, generator 200 may activate or deactivate cooling, and/or modify the cooling rate in response to change in mode, for example switching from a monopolar mode to a bipolar mode. Generator 200 may also turn cooling on or off when RF energy delivery is started or stopped.

In some embodiments, system 1000 comprises one or more switches associated with the radiofrequency ablation device 100, generator 200, and/or part of a separate device, for example a foot pedal, (not shown).

In some embodiments, radiofrequency ablation device 100 may include irrigation features (not shown) where a fluid is ejected through one or more orifices into or near the target tissue. The fluid may be the same fluid used to cool the probe.

### Automatic Injection—Temperature Mode and Bolus Mode

In one embodiment, system 1000 includes an ultrasound system (not shown) connected to generator 200. Ultrasound system can be any known system including a combination of devices (transducer, user interface, control panel, display, processor, etc.) that use sound waves to view inside the body and produce images on a display.

In use, a distance between structures can be measured, for example using artificial intelligence software, and fluid can be injected based on measured distances.

### Method of Ablating a Thyroid Nodule and Injecting Fluid in Response to a Temperature

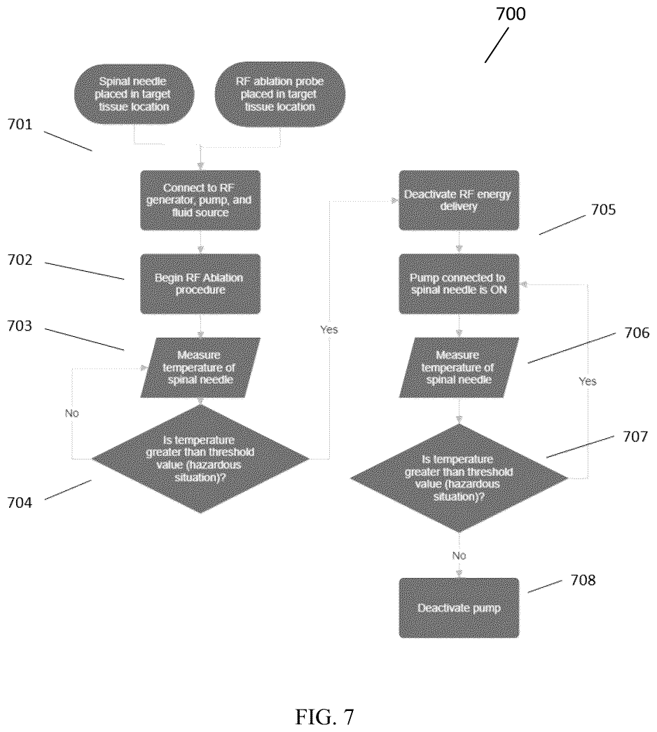

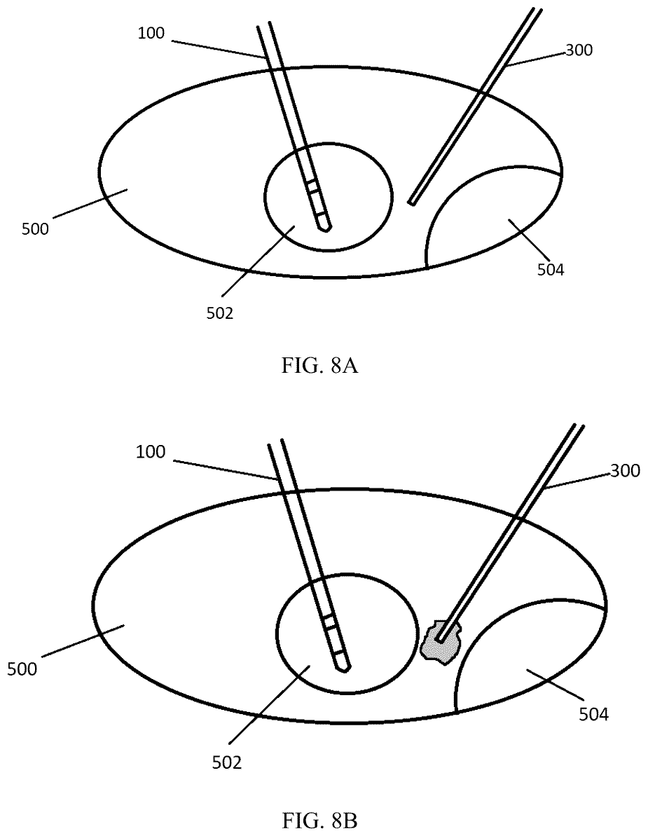

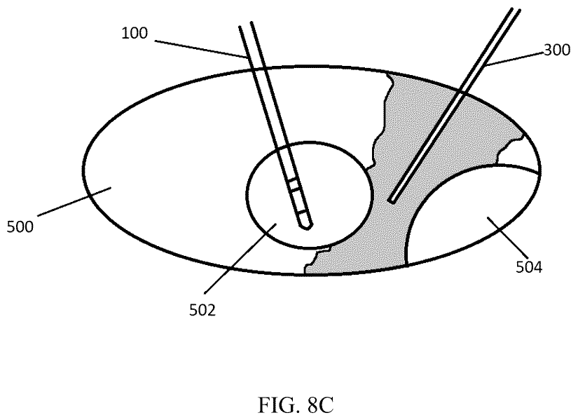

FIG. 7 illustrates a method for ablating a tissue using the tissue ablation system 1000 described herein. By way of example only, the procedure described involves ablating a thyroid nodule, however the same method may be used to ablate other types of (soft) tissue, for example, any ablation procedure where the ablation is intended to be created in proximity to other structures that are not to be ablated, such as the DRG when ablating the facet joint in pain management procedures; the spinal canal when performing spinal tumor ablation; motor nerves when performing genicular nerve ablation; esophageal tissue when performing cardiac ablation procedures. The method may be better understood with reference to FIGS. 8a to 8c.

Method 700 begins at step 701. The fluid injection device (needle) 300 and radiofrequency ablation device 100 are connected to the RF generator 200, and pump 400. The pump 400 is connected to a fluid source. Using a known imaging technique, the radiofrequency ablation device 100 is inserted into the thyroid 500, (through the isthmus), and into the thyroid nodule 502. Fluid injection device 300 is also inserted into thyroid 500, for example, such that the distal end is located between thyroid nodule 502 and a sensitive tissue portion 504, as shown in FIG. 8A.

Fluid injection device 300 includes at least one temperature sensor located in the distal region. Sensitive tissue portion 504 contains one or more sensitive structures and may include a portion of thyroid and or structures outside of the thyroid. Sensitive structures include, for example, the recurrent laryngeal nerve, esophagus, carotid artery, vocal cords. One or more such structures may be located and what is known as the “danger triangle” to those skilled in the art.

At step 702, a physician begins the ablation procedure and delivers radiofrequency energy to thyroid nodule 502. RF energy may be delivered in either a monopolar mode or a bipolar mode, as described herein. In some embodiments, a physician switches between monopolar mode and bipolar mode in the same procedure, depending on the configuration of the radiofrequency ablation radiofrequency ablation device 100.

At step 703, while RF energy is being delivered, thyroid tissue around nodule 502 heats up, and temperature sensor 318 measures a temperature within the tissue, for example, between thyroid nodule 502 and the sensitive tissue portion 504. The measured temperature is sent to generator 200.

At step 704, generator 200 receives the measured temperature from temperature sensor 318 and compares it to a threshold temperature value. In one example, the threshold temperature value is 42 °C. Ablation continues as long as the measured temperature remains in a safe range, for example, below the threshold temperature. In such a case, steps 702 and 703 continue cycling until the ablation is complete.

If the measured temperature value received from temperature sensor 318 is greater than the threshold temperature, the method proceeds to step 705.

At step 705, generator 200 deactivates RF energy in the probe, and instructs the fluid delivery device 400 to pump fluid to the fluid injection device 300, which in term, is injected to the thyroid tissue, between the nodule 502 and the sensitive tissue portion 504, as shown in FIG. 8B.

At step 706, as fluid is being injected, fluid injection device 300 continues to measure a temperature and send it to the generator.

At step 707, generator 200 continues to check the measured temperature against a threshold value. Pumping of fluid continues until the measured temperature decreases below the threshold temperature. In some cases, fluid may form a thermal or electrical barrier between thyroid nodule 502 and sensitive tissue portion 504, as shown in FIG. 8C.

When the measured temperature decreases below the threshold, the method proceeds to step 708, and the pump is deactivated. At this point the RF ablation procedure can be resumed, i.e., return to step 702, with the RF probe at the same location or a different location within the thyroid nodule 502.

During the procedure, fluid may dissipate, decreasing the size of the barrier, and may disappear altogether. Any of steps 702 to 708 may be repeated if and when the measured temperature surpasses the threshold temperature.

The physician may use any number of monitoring parameters to determine when the ablation is complete. A physician may use known imaging techniques or modalities. Additionally, or alternatively, acoustic properties or echogenic changes may be measured by ultrasound. In other examples, RF energy can be delivered for a predetermined time, or may be delivered until a measured impedance increases to a known amount, or after a certain number of Joules have been delivered.

The physician may move radiofrequency ablation 100 to other locations within the nodule and repeat any steps in method 700 until the nodule 502 is substantially ablated, as determined by the physician. The procedure ends when a significant portion of the nodule 502 has been ablated, for example when a certain percentage of the tissue has been ablated. radiofrequency ablation device 100 and fluid injection device 300 are then removed from the tissue.

In some embodiments, cooling features described herein are used to cool a portion of the radiofrequency ablation device 100. For example, cooling may be used during RF delivery and automatically halted when RF energy delivery ceases, for example at step 705.

### Method of Ablating a Thyroid Nodule and Injecting Fluid in Response to a Measured Distance (Bolus Mode)

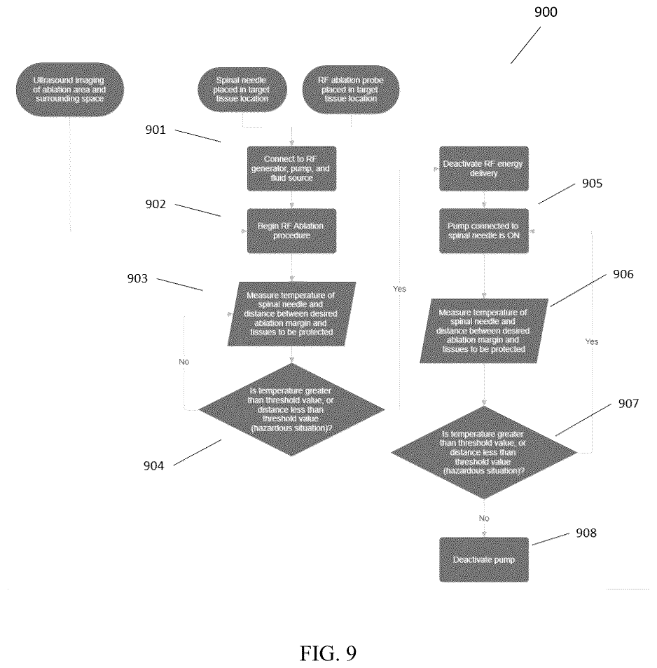

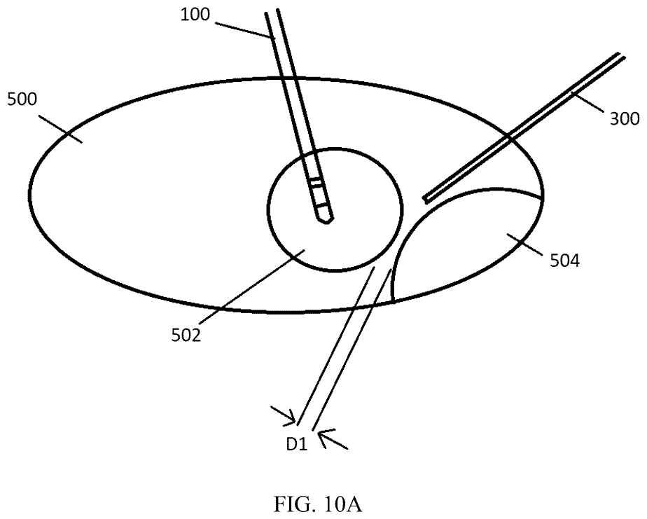

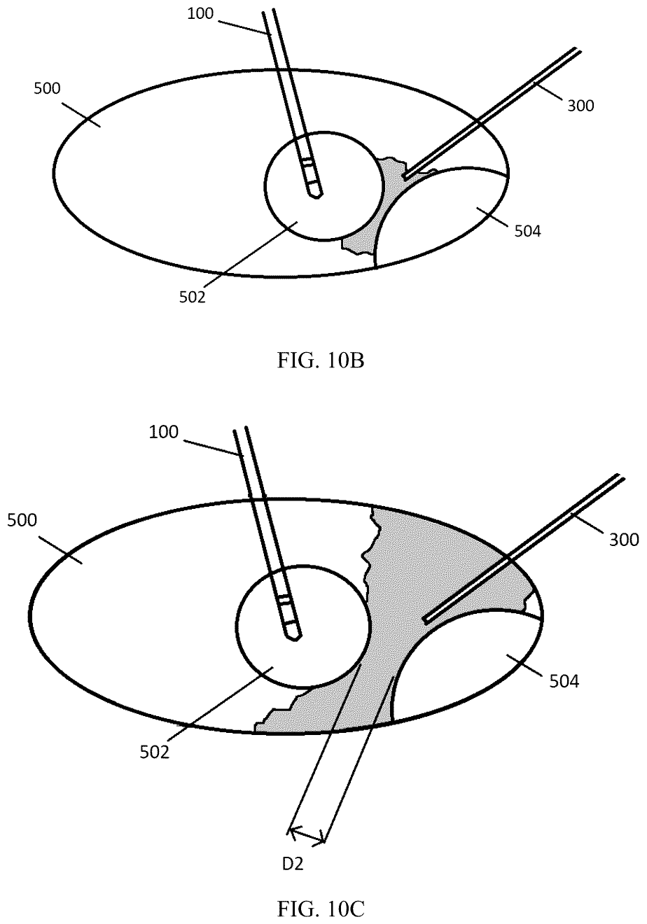

FIG. 9 illustrates a method for ablating a tissue using the tissue ablation system 1000 described herein, and comprising an imaging system, for example ultrasound imaging. By way of example only, the procedure described involves ablating a thyroid nodule, however the same method may be used to ablate other types of tissue, described herein. The method may be better understood with reference to FIGS. 10A to 10C. Aspects of method 900 are similar to method 700 described herein.

Method 900 begins at step 901. The fluid injection device 300 and radiofrequency ablation device 100 are connected to the generator 200, and fluid delivery device 400, which is in turn connected to a fluid source. Using a known imaging technique, the radiofrequency ablation device 100 is inserted into the thyroid nodule 502. Fluid injection device 300 is also inserted into thyroid 500, for example, such that the distal end is located between thyroid nodule 502 and a sensitive tissue portion 504, as shown in FIG. 10A. In one embodiment, the imaging technique is ultrasound.

Prior to delivery of any RF energy, thyroid nodule 502 may be located a distance D1 from sensitive tissue portion 504. For clarity, distance D1 may be any measurable distance between any portion of nodule 502 and the sensitive tissue portion 504. For example, the closest distance between two points on each tissue portion, or an average distance.

In some embodiments, fluid injection device 300 includes at least one temperature sensor located in the distal region.

At step 902, a physician begins the ablation procedure and delivers radiofrequency energy to thyroid nodule 502.

At step 903, while RF energy is being delivered, thyroid tissue around nodule 502 heats up, and temperature sensor 318 measures a temperature within the tissue, for example, between thyroid nodule 502 and the sensitive tissue portion 504. The measured temperature is sent to generator 200.

Additionally, a distance between the structure may be observed by imaging, and in some examples, that distance is sent to the generator.

At step 904, in some examples, generator 200 receives the measured temperature from temperature sensor 318 and compares it to a threshold temperature value. Additionally, the generator compares the distance D1, along with the measured temperature.

Any combination of temperature and distance may be used to determine a hazard condition threshold. For example, depending on the distance, a higher or lower threshold temperature may be used.

Ablation continues as long as a hazard condition is not detected. In such a case, steps 902 and 903 continue cycling until the ablation is complete. If a hazard condition is detected, the method proceeds to step 905.

At step 905, generator 200 deactivates RF energy in the probe, and instructs the fluid delivery device 400 to pump fluid to the fluid injection device 300, which in term, is injected to the thyroid tissue, between the nodule 502 and the sensitive tissue portion 504, as shown in FIG. 10B.

At step 906, as fluid is being injected, fluid injection device 300 continues to measure a temperature and send it to the generator. The distance between the structures is also sent to the generator.

At step 907, generator 200 continues to check the measured temperature and a distance to determine if the hazard condition is present. Pumping of fluid continues until the hazard condition is absent. In some cases, fluid may form a thermal or electrical barrier between thyroid nodule 502 and sensitive tissue portion 504, as shown in FIG. 10C. In some cases, the distance D2 between the two structures has increased.

When the hazard condition is no longer present, the method proceeds to step 908, and the pump is deactivated. At this point the RF ablation procedure can be resumed, i.e., return to step 902, with the RF probe at the same location or a different location within the thyroid nodule 502.

During the procedure, fluid may dissipate, decreasing the size of the barrier, and may disappear altogether. Any of steps 902 to 908 may be repeated if and when a hazard condition is detected.

The physician may use any number of monitoring parameters to determine when the ablation is complete. The physician may move radiofrequency ablation device 100 to other locations within the nodule and repeat any steps in method 900 until the nodule 502 is substantially ablated. The procedure ends when a significant portion of the nodule 502 has been ablated. Radiofrequency ablation device 100 and fluid injection device 300 are then removed from the tissue.

In some embodiments, cooling features described herein are used to cool a portion of the radiofrequency ablation device 100. For example, cooling may be used during RF delivery and automatically halted when RF energy delivery ceases, for example at step 905.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention.

1. A method of delivering energy to a region of tissue within a patient's body in conjunction with controlled fluid delivery, the method comprising the steps of:
monitoring a tissue parameter within the region of tissue;
detecting whether a value of the tissue parameter is within an allowable range;
while the tissue parameter remains within the allowable range, delivering radiofrequency energy to a first portion of tissue within a region of tissue; and
if the tissue parameter exits the allowable range, automatically delivering fluid to the region of tissue.

2. The method of claim 1, wherein fluid is delivered between the first portion of tissue within the region of tissue and a second portion of tissue within the region of tissue

3. The method of claim 1, wherein the tissue parameter is a temperature and wherein the allowable range is less than 42 degrees Celsius.

4. The method of claim 2, wherein the tissue parameter is a distance between the first portion of tissue and the second portion of tissue.

5. The method of claim 1, wherein the step of delivering radiofrequency energy is performed using a first device and step of injecting fluid is performed using a second device.

6. The method of claim 1, wherein the fluid is delivered for a predetermined amount of time.

7. The method of claim 1, wherein delivering fluid includes delivering a predetermined volume of fluid.

8. The method of claim 1, wherein the region of tissue is a thyroid, and the first portion of tissue is a thyroid nodule.

9. The method of claim 1, wherein the tissue parameter is a first tissue parameter, the value is a first value, and the allowable range is a first allowable range, the method further comprises the steps of:
monitoring a second tissue parameter within the region of tissue;
detecting whether a second value of the second tissue parameter is within a second allowable range;
while the second tissue parameter remains within the second allowable range, delivering radiofrequency energy to the first portion of tissue within a region of tissue; and
if the second tissue parameter exits the second allowable range, automatically delivering fluid to the region of tissue.

10. A system for controlling delivery of radiofrequency energy and controlling delivery of fluid to a region of tissue comprising:
an electrosurgical device for delivering radiofrequency energy;
a fluid injection device;
a fluid delivery device; and
a generator in communication with the electrosurgical device, the fluid injection device, and the fluid delivery device;
wherein the generator monitors at least one tissue parameter, and wherein the generator controls delivery of radiofrequency energy to the electrosurgical device and controls delivery of fluid to the fluid injection device in response to a change in a value of the at least one tissue parameter.

11. The system of claim 10, wherein the electrosurgical device comprises an elongate member defining a lumen for circulating a cooling fluid, and wherein the generator controls circulation of the cooling fluid.

12. The system of claim 10, wherein the electrosurgical device comprises at least one temperature sensor.

13. The system of claim 10, wherein the fluid injection device comprises a temperature sensor, and the at least one tissue parameter is a temperature value.

14. The system of claim 10 further comprising an ultrasound imaging device, and the at least one tissue parameter is a distance between a first portion of tissue within the region of tissue and a second portion of tissue within the region of tissue, wherein the distance is measured using ultrasound imaging.

15. The system of claim 10 further comprising a fluid deactivation mechanism.
